(19) United States
(12) Patent Application Publication
(10) Pub. No.: US 2007/0077270 A1
(43) Pub. Date: Apr. 5, 2007

(54) Delivery devices and methods for long-term, targeted delivery of therapeutic agents to the eye and ear

(75) Inventor: Xuejun Wen, Mount Pleasant, SC (US)

Correspondence Address: DORITY & MANNING, P.A., POST OFFICE BOX 1449, GREENVILLE, SC, 29602-1449, US

(73) Assignee: Clemson University

(21) Appl. No.: 11/390,958

(22) Filed: Mar. 28, 2006

Related U.S. Application Data
(60) Provisional application No. 60/665,711, filed on Mar. 28, 2005.

(51) Int. Cl. A61F 2/02

(52) U.S. Cl. 424/423; 424/427

(57) ABSTRACT

Disclosed are devices and methods for targeted delivery of therapeutic agents. The devices include selectively permeable hollow fiber membranes which allow for the outward diffusion of therapeutic agents while the contents of the device are protected from host humoral and cellular immunologic attack. The methods include implanting the devices in the ears and/or eyes.

### CROSS REFERENCE TO RELATED APPLICATION

The present application claims benefit of United States Provisional Application Ser. No. 60/665,711 having a filing date of Mar. 28, 2005.

### BACKGROUND

In treating disorders of the ear and eye, it can be desirable to deliver therapeutic agents in a targeted, safe, and efficient manner. However, the targeted delivery of agents to the ear and eye can present many challenges.

Loss of hearing function due to heredity, aging, or pathologies in the auditory system often results in disabilities in independence, communication, and lifestyle. Statistical data from NIH/NIDCD show that there are approximately 28 million Americans with hearing impairments. Hearing loss affects approximately 17 in 1,000 children under age 18, and the incidence increases with age. Approximately 314 in 1,000 people over age 65 and 40 to 50 percent of people of age 75 or older have a hearing loss. Thus, hearing loss poses a major health care burden for our society, and there is a compelling need for effective interventional therapies for auditory disorders.

Current therapies to treat ear disorders are largely dependent upon intra-ear delivery of therapeutic agents. The efficacy of delivered agents in retarding disease progression, alleviating symptoms, and hastening functional recovery is well known. However, cessation of agent delivery often results in an exacerbation of disease as compared to untreated conditions, suggesting a critical need of long-term sustained delivery with unlimited temporal profile.

In most of the available delivery strategies, repeated trans-tympanic blind injections or agent refillings are necessary to maintain the local concentration of the agent in the diseased ear. These procedures significantly decrease patient compliance and increase risks of infection and inflammation.

More recently, the emergence of intra-ear perfusion delivery strategies has offered new treatments for auditory damages and disease. However, these approaches have been found problematic with regard to uneven delivery profiles, limited temporal delivery profiles, and retrieval difficulties.

When considering patients requiring cochlear implants, for instance in the treatment for deafness caused by hair cell loss, one major problem has been the secondary degeneration of auditory neurons over time due to the lack of endogenous neurotrophin supply from normal hair cells. Such peripheral degeneration can then lead to loss of central auditory nuclei and successive impairments of auditory function. In order to prevent the secondary auditory neuron degeneration, the long-term delivery of neurotrophins to the surrounding neurons from a cochlear implant is necessary. To this end, a cochlear implant from which neurotrophins can be continuously delivered is highly desirable.

Similarly, current therapies to treat eye disorders are largely dependent on classic methods of ocular drug delivery. Even the least invasive, i.e. topical treatment, can still cause significant systemic effects due to, e.g., absorption of therapeutic materials by the nasolacrimal duct and nasopharynx. Systemic treatments, in which drugs can be widely distributed throughout the body, can result in unwanted effects as well. Treatment by periocular injection often has a limited effect on the target tissue because drugs must cross the blood-ocular barrier. Intravitreal injections are most effective, but often require multiple injections, thereby increasing patient discomfort, cost, and risk of side effects. In addition, other problems with current delivery approaches include inability to refill an implanted delivery device, difficulty in retrieval of an implanted delivery device, and inability to alter the type of delivered agents.

### SUMMARY

The present disclosure recognizes and addresses the foregoing needs as well as others in the treatment of ear and eye diseases. In one embodiment of the present disclosure, a device for delivery of therapeutic agents is provided. The device includes a body having a proximal end and a distal end and defining a cavity. An access port is located at the proximal end of the body and a removable insert is configured to be removably inserted into the cavity of the body.

In certain embodiments, the removable insert may include one or more therapeutic agents. The body may be configured for use in proximity to the ear and/or eye. The body may comprise a selectively permeable hollow fiber membrane. The selectively permeable hollow fiber membrane may have less than 200 KDa molecular weight cut off. The selectively permeable hollow fiber CO membrane may have less than 70 KDa molecular weight cut off. The selectively permeable hollow fiber membrane may have less than 40 KDa molecular weight cut off. The selectively permeable hollow fiber membrane may have less than 20 KDa molecular weight cut off. The access port may be formed from a biocompatible flexible polymer. The access port may include a cap configured to removably engage the access port such that no therapeutic agent can exit the body through the access port. The removable insert may include a selectively permeable hollow fiber membrane. The removable insert may include a degradable rod configured to dissolve and release therapeutic agent.

In another embodiment of the present disclosure, a method for targeted delivery of therapeutic agent to the inner ear is provided. The method includes providing a device having a body, an access port, and a removable insert. The body includes a selectively permeable hollow fiber membrane and the removable insert includes one or more therapeutic agents. The device is implanted whereby the therapeutic agent is delivered to an inner ear.

In still another embodiment of the present disclosure, a method for targeted delivery of therapeutic agent to the eye is provided. The method includes providing a device having a body, an access port, and a removable insert. The body includes a selectively permeable hollow fiber membrane and the removable insert includes one or more therapeutic agents. The device is implanted whereby the therapeutic agent is delivered to an eye.

### DESCRIPTION OF THE DRAWINGS

A full and enabling disclosure, including the best mode thereof to one of ordinary skill in the art, is set forth more particularly in the remainder of the specification, including reference to the accompanying figures in which:

FIGS. 1A and 1B illustrate the delivery device in accordance with different embodiments of the present disclosure;

FIGS. 2A and 2B illustrate the re-sealable access port and cap of the delivery device in accordance with different embodiments of the present disclosure;

FIGS. 3A and 3B illustrate the body of the delivery device in accordance with different embodiments of the present disclosure;

FIGS. 4A, 4B, and 4C illustrate removable inserts for delivery of therapeutic agents in accordance with different embodiments of the present disclosure;

FIGS. 5A and 5B illustrate intra-ear placement of the delivery device in accordance with one embodiment of the present disclosure;

FIG. 6A illustrates the body of the delivery device in accordance with one embodiment of the present disclosure;

FIG. 6B illustrates a removable insert for delivery of therapeutic agents in accordance with one embodiment of the present disclosure;

FIG. 6C illustrates the delivery device in accordance with one embodiment of the present disclosure;

FIG. 6D illustrates intra-ear placement of the delivery device in accordance with one embodiment of the present disclosure; and

FIG. 7 illustrates transcleral placement of the delivery device in accordance with one embodiment of the present disclosure.

Repeat use of reference characters in the present specification and drawings is intended to represent the same or analogous features or elements of the present disclosure.

### DETAILED DESCRIPTION

Reference will now be made in detail to the embodiments of the disclosure, examples of which are illustrated in the accompanying drawings. While the a disclosure will be described in conjunction with the preferred embodiments, it will be understood that they are not intended to limit the disclosure to these embodiments. On the contrary, the disclosure is intended to cover alternatives, modifications and equivalents, which can be included within the spirit and scope of the disclosure as defined by the appended claims. Furthermore, in the following detailed description of the present disclosure, numerous specific details are set forth in order to provide a thorough understanding of the present disclosure. However, it will be obvious to one of ordinary skill in the art that the present disclosure can be practiced without these specific details. In other instances, well-known methods, procedures, and components have not been described in detail as not to unnecessarily obscure aspects of the present disclosure.

In general, the present disclosure is directed to devices and methods for targeted delivery of therapeutic agents, and in certain embodiments, for delivery to the ear and/or eye. One advantage of the devices is that they can be refillable following implantation so as to allow for unlimited delivery periods and amounts of materials. In addition, the devices can be refilled following implantation with either the same or different types of therapeutic agents. Moreover, the devices can be refilled without causing damage to the host tissue or the devices. Likewise, therapeutic agents contained in the devices can be easily removed if necessary without causing damage to the host tissue or the devices.

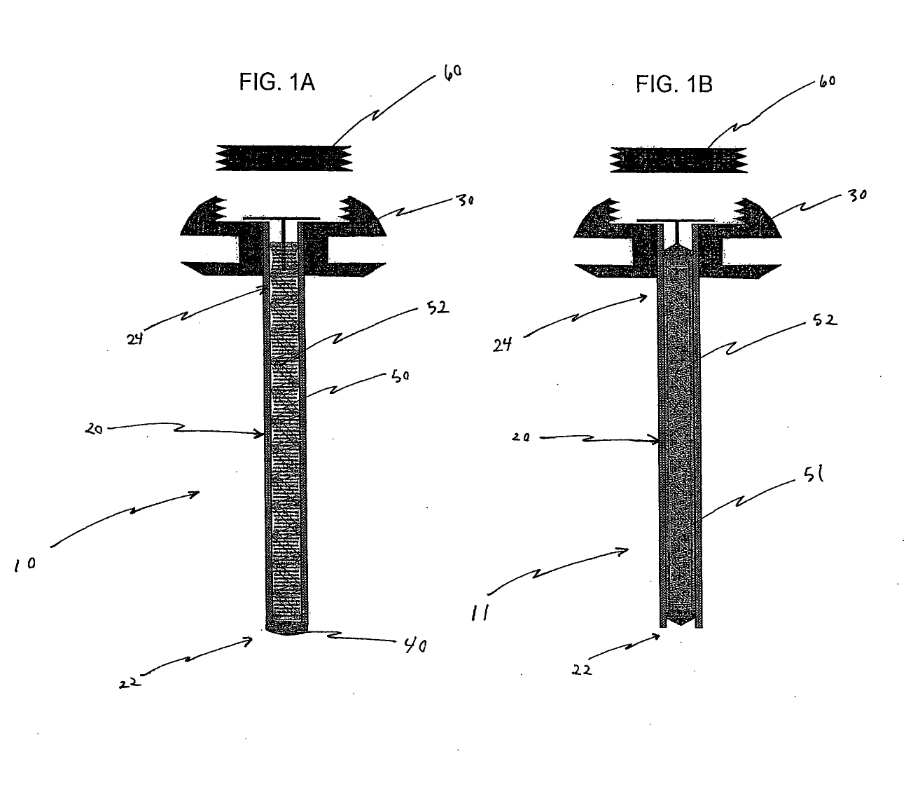

Referring to FIGS. 1A and 1B, delivery devices 10, 11 in accordance with the present disclosure are shown. For example, each delivery device 10 can include a body 20 formed from a membrane. In some embodiments, the body 20 can be formed from a selectively permeable hollow fiber membrane. A selectively permeable hollow fiber membrane allows for a uniform delivery profile of therapeutic agent. In one embodiment, a selectively permeable hollow fiber membrane with less than 200 KDa molecular weight cut off (MWCO) can be utilized. For example, in one embodiment, a selectively permeable hollow fiber membrane of 70 KDa MWCO can be utilized. In still another embodiment, a selectively permeable hollow fiber membrane of 40 KDa MWCO can be utilized. In yet another embodiment, a selectively permeable hollow fiber membrane of 20 KDa MWCO can be utilized.

Accordingly, a selectively permeable hollow fiber membrane having a 40 kDa MWCO can allow diffusion of therapeutic agents 52 including agents secreted from cells while the contents of the delivery device 10 (such as encapsulated cells or tissues in some embodiments) are protected from host humoral and cellular immunologic attack (most neutrotrophins are less than 40 KDa, most hormones are less than 20 KDa, most drugs and vitamins are less than 1 KDa, while most humoral immune components are larger than 140 KDa).

According to the present disclosure, the term “selectively permeable hollow fiber membrane” refers to a porous polymeric structure that can selectively allow molecules less than the size of the pores to pass through the membrane. In some embodiments, selectively permeable hollow fiber membranes can be fabricated using a wet phase inversion technique although any other method as would be known in the art can also be utilized.

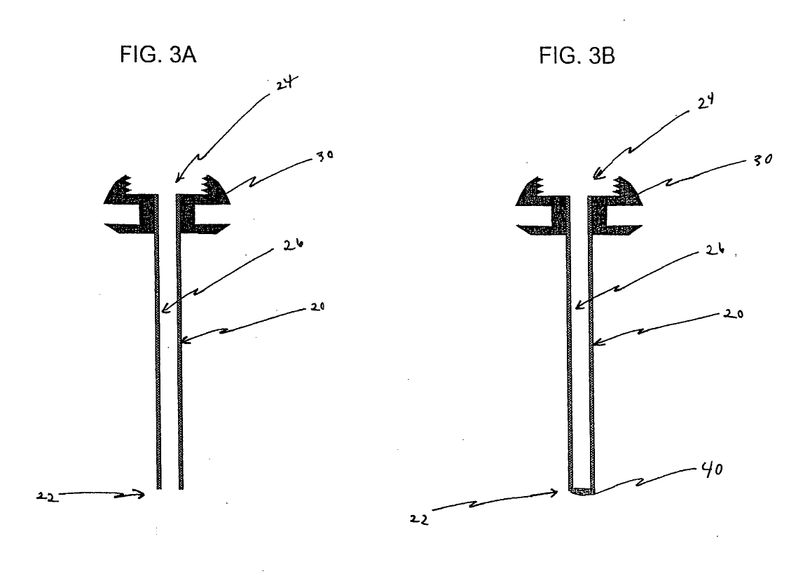

As illustrated in FIGS. 3A and 3B, in certain embodiments, the body 20 of the delivery device 10 can have a generally linear, cylindrical shape with a distal end 22 and proximal end 24. However, the body 20, is not limited to a generally linear, cylindrical shape and other non-linear shapes can be utilized as well. The body 20 can have an internal volume suitable to hold a volume of therapeutic agent 52 within the lumen 26. In addition, the surface area of the body 20 can be large enough as to provide a sufficient area through which therapeutic agent 52 can be delivered. In some embodiments, permeability of the body 20 can be varied along the length such that proximal end 24 is less permeable than the distal end 22 of the device or vice versa.

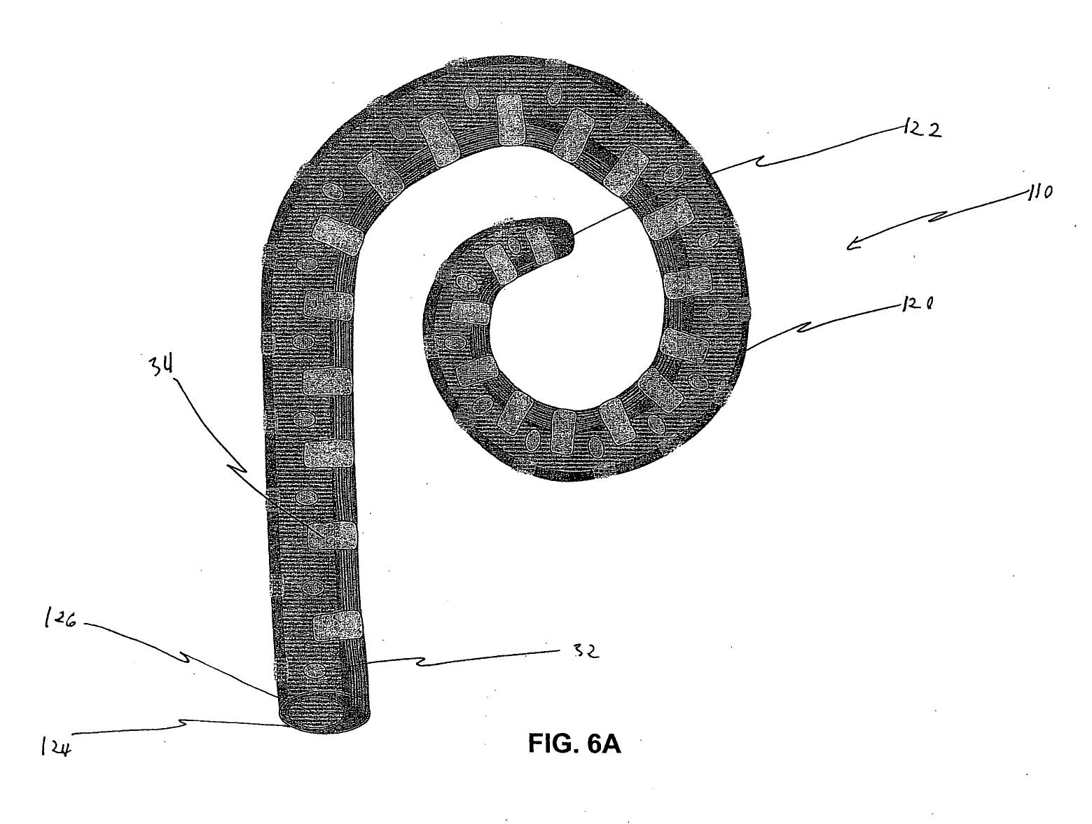

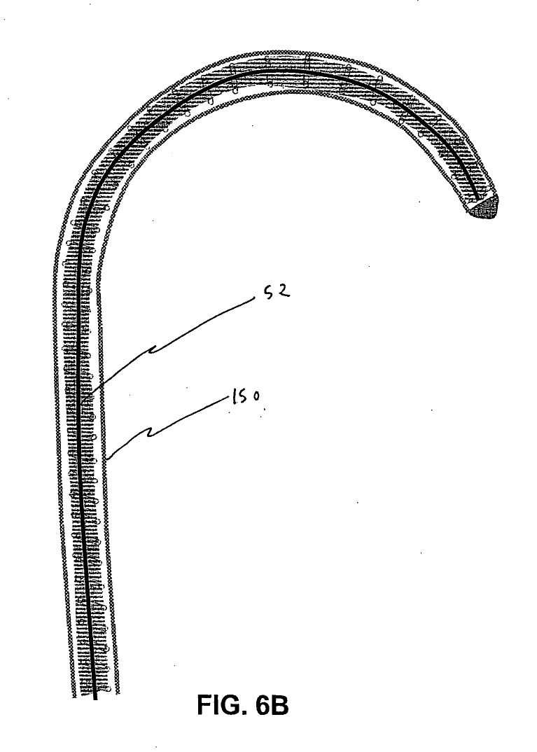

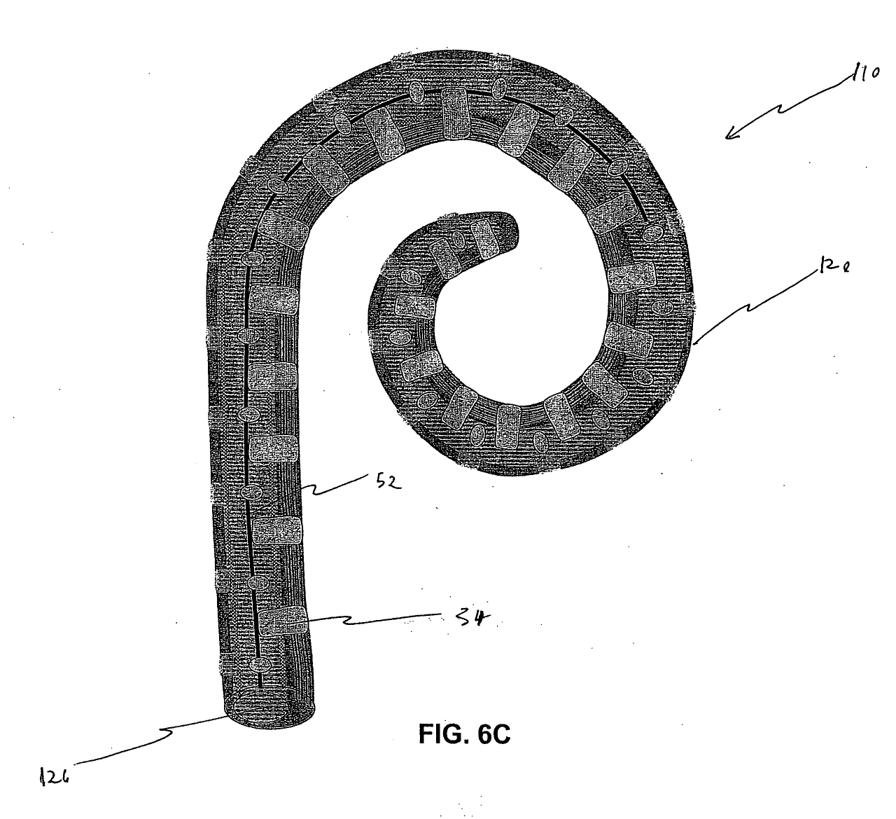

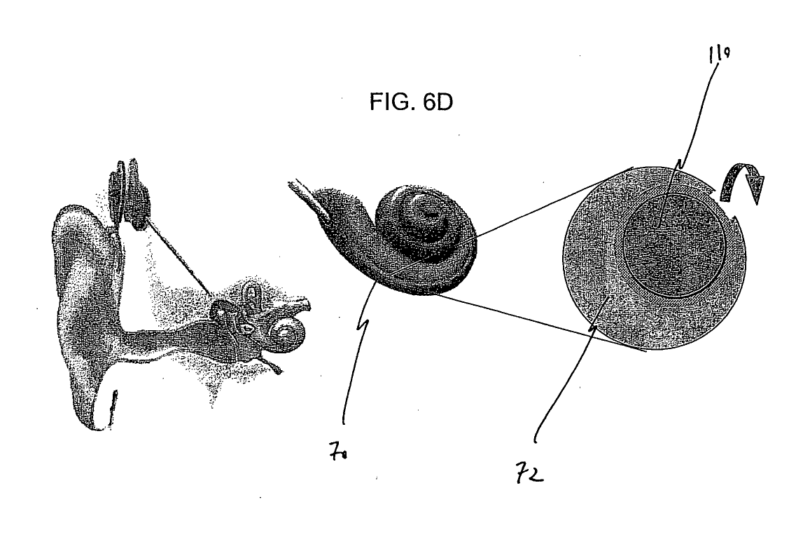

Referring to FIG. 6A, an alternative embodiment of a delivery device 110 is illustrated. In one embodiment, such a device 110 can be a cochlear implant with therapeutic agent 52 inside the implant. According to this particular embodiment, the device 110 can have a hollow-core cochlear implant body 120. The body 120 can be formed from any suitable material as would be known in the art such as any biocompatible polymers including polyurethane, polypropylene, or the like. As illustrated in FIG. 6A, the body 120 of the delivery device 110 can have a generally spiral, cylindrical shape with a distal end 122 and proximal end 124. The body 120 can have an internal volume suitable to hold a volume of therapeutic agent 52 within the lumen 126 and which can also provide a sufficient surface area through which therapeutic agent 52 can be delivered. The body 120 can also define orifices 32 through which therapeutic agent 52 can be released. In some embodiments, the orifices 32 can be less than 100 microns in diameter. In some embodiments, the orifices can be less than 50 microns. In some embodiments, the orifices can range between 0.01 microns and 1 micron in diameter. Such orifices 32 can be fabricated in the device using a laser micro-fabrication facility or other methods as would be known in the art. In some embodiments, the body 120 can also have stimulating contacts 34. Stimulating contacts are electrodes to stimulate auditory nerves to generate sound signals in patients.

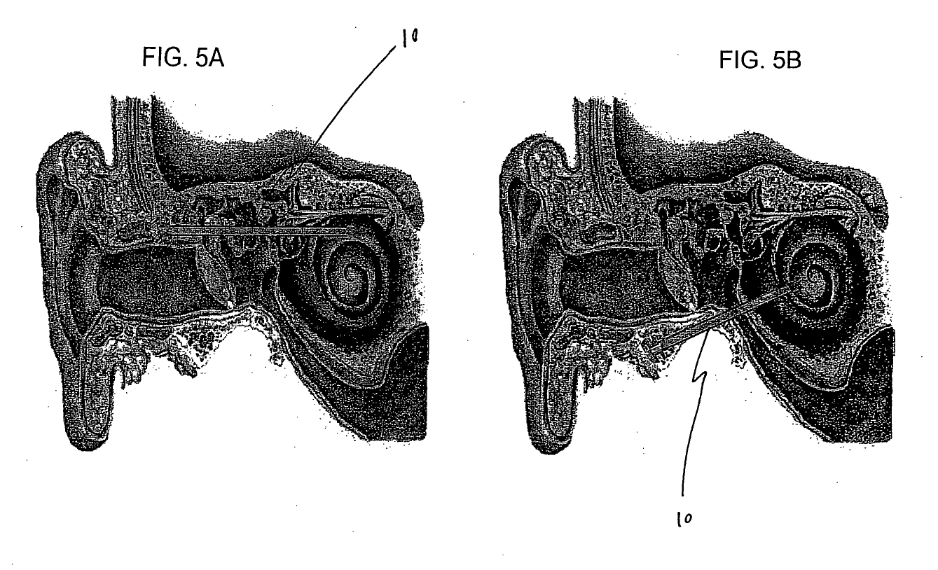

Referring again to FIG. 1, in one embodiment, a re-sealable access port 30, can be located on the proximal end 24 of the body 20 of delivery device 10. In one embodiment, the access port 30 can be formed from a biocompatible flexible polymer, such as an elastomer. For example, the access port 30 can be formed from a flexible polyurethane. In this regard, biocompatible refers to a material that is substantially non-immunogenic. The overall size and shape of the access port 30 is not particularly limited. In one embodiment, the access port 30 can also serve as an anchor to maintain the body 20 at the site of implantation. According to this embodiment, access port 30 can be of a size sufficient to anchor the body 20 of the delivery device 10 to the general area where delivery of therapeutic agent 52 is desired. In some embodiments, the access port 30 is less than 5 millimeters in length. In some embodiments, the access port 30 is less than 2 millimeters in length. In some embodiments, the access port 30 is less than 1 millimeter in length. In some embodiments, the access port 30 can be anchored into the temporal bone (see, e.g. FIG. 5A and 5B). The access port 30 can allow for unlimited agent-loading, replacement, and retrieval, to the lumen 26 without damage to the surrounding tissue or device 10. Beneficially, only a small skin incision with local anesthesia can be required to access the access port 30, depending on the specific location of the device 10. In other embodiments, access port 30 can be directly accessible, with little inconvenience to a patient.

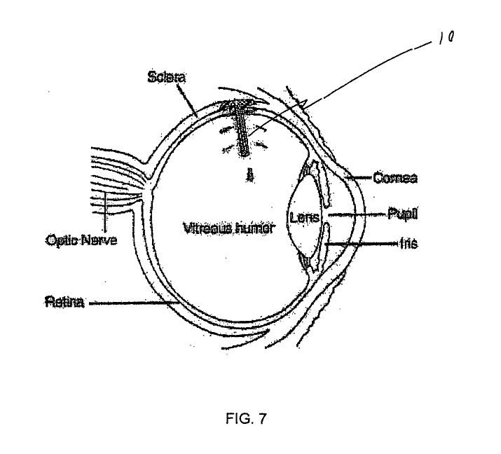

FIG. 7 illustrates transcleral placement of a delivery device 10, in which the access port 30 can be anchored at the pars plana of the eye. It should be understood, however, that this is an exemplary embodiment only, and in other embodiments, the delivery device 10 can be placed in other specific locations. For purposes of this disclosure, ocular region refers to the eye, including all its muscles, nerves, blood vessels, tear ducts, membranes, as well as structures that are immediately adjacent to the eye and its physiological functions.

In another embodiment, the disclosed devices can be utilized for delivery of prophylactic, therapeutic, or any other suitable biologically active agents in the otic region. Placement of the delivery device 10 in or near the ear (one embodiment of which is illustrated in FIG. 5A and 5B) can be at any suitable location in the otic region. For purposes of this disclosure, otic refers to the ear including but not limited to the external ear, middle ear, cochlea, the endolymphatic sac/duct, the vestibular labyrinth, and all of the compartments/connecting tubes that include or contain any of these components. In some embodiments, the delivery device 10 can be implanted in conjunction with one or more additional implantation devices as is known in the art. For example, in one preferred embodiment, the delivery device 10 can be incorporated into a cochlear implant (one embodiment illustrated in FIG. 5A) or as a new device (one embodiment illustrated in FIG. 6D).

Referring to FIG. 1A, in certain embodiments of the present disclosure, the distal end 22 of the body 20 can be sealed with medical grade adhesive seal 40 or the like. The seal 40 can be shaped with any geometry, for example pointed, or blunt, or any other suitable shape. Seal 40 can serve, for example, to seal the body 20 of the delivery device 10 and prevent therapeutic agent 52 from exiting the device from the disfal end 22 and thus encourage exit of therapeutic agent 52 through the body wall, for example through the wall of a selectively permeable hollow fiber membrane of the body 20. However, as illustrated in FIGS. 1B and 3A, the distal end 22 of the body 20 can also be open to allow for high volume delivery of therapeutic agent 52. Accordingly, in certain embodiments, the wall of body 20 can be impermeable.

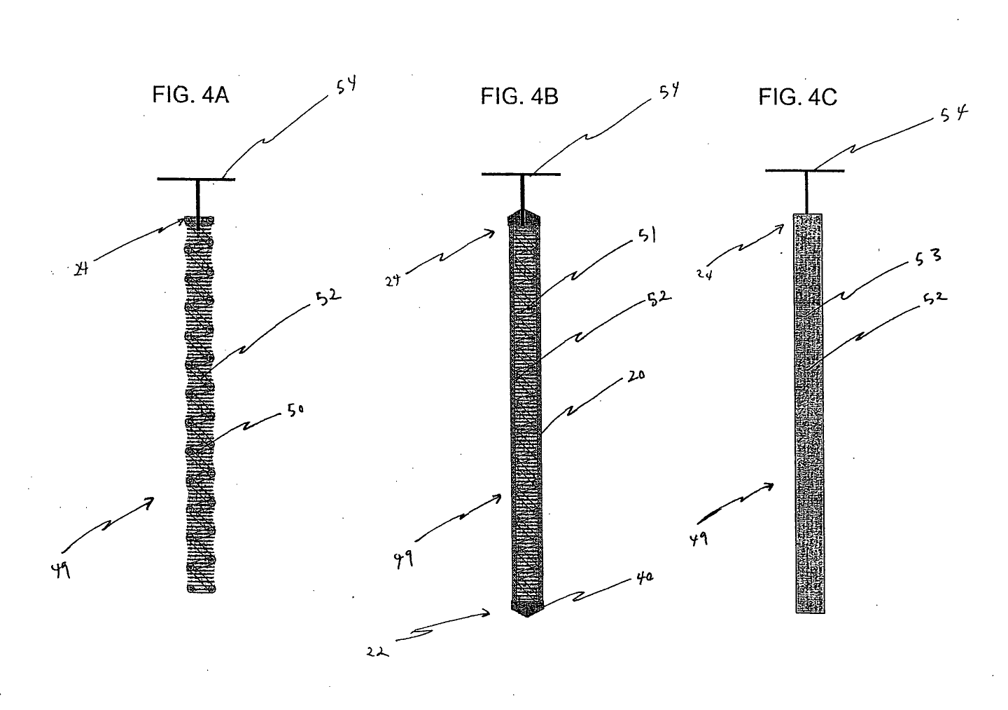

FIGS. 4A-4C illustrate different embodiments of inserts 49 for delivery of therapeutic agents to the devices of the present disclosure. In certain embodiments, such inserts 49 are removable. In certain embodiments, such inserts 49 are positioned inside the body 20 of the delivery device 10. In certain embodiments, the insert can include biodegradable synthetic polymeric scaffold materials such as, for example, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprolactone, polyphophazenes, polyorthoesters, polyglycolide, copolymers of lysine and lactic acid, copolymers of lysine-RGD and lactic acid, and the like, and copolymers of the same. Optionally, the insert can include naturally derived biodegradable materials including, but not limited to chitosan, agarose, alginate, collagen, hyaluronic acid, and carrageenan (a carboxylated seaweed polysaccharide), demineralized bone matrix, and the like, and copolymers of the same. Optionally, the insert can include factors that can be released as the scaffold(s) degrade. For example, the anchorage can include one or more factors that can trigger one or more cellular events. According to this embodiment, as the scaffold(s) forming the cellular anchorage degrades, the factors can be released and interact with the cells.

For example, in the embodiment shown in FIG. 4A, a coil-based vehicle 50 can be designed to escort therapeutic agent 52 into or out of the hollow fiber membrane lumen 26. The coil-based vehicle 50 can resemble a screen structure that allows therapeutic agent 52 to be anchored to the vehicle 50 for delivery into the body 20 of the delivery device 10. The coil-based vehicle can be formed from a biocompatible polymer, metal, composite, or any other suitable material as would be known to one of ordinary skill in the art.

In another embodiment, as shown in FIG. 4B, a semi-permeable hollow fiber membrane capsule 51 can be designed to escort therapeutic agent 52 into or out of the hollow fiber membrane lumen 26. The hollow fiber membrane capsule 51 can allow for additional control of release of therapeutic agent 52 by adding an additional hollow fiber membrane layer. In such embodiments, the release of therapeutic agent 52 can be further controlled by the addition of such a layer. In addition, the hollow fiber membrane capsule. 51 can have a seal 40 on the distal end 22 of the capsule 51. The seal 40 can serve to prevent therapeutic agent 52 from exiting the capsule 51 through the distal end 22, but rather encourage exit through the hollow fiber membrane wall of the capsule 51.

In yet another embodiment, as shown in FIG. 4C, a solid rod 53 can be designed to escort therapeutic agent 52 into or out of the hollow fiber membrane lumen 26. The rod 53 can provide for a more long-term delivery rate. For example, the rod 53 can degrade or completely dissolve within the body 20 of the delivery device 10 and release therapeutic agent 52 from the rod 53.

In still another embodiment, as shown in FIG. 6B, a coil-based vehicle 150 is illustrated that can escort therapeutic agent 52 into or out of the lumen 26 of the body 20 (see FIG. 6C). The coil-based vehicle 150 resembles a screen structure that allows therapeutic agent 52 to be anchored to the vehicle 150 for delivery into the body 20 of the delivery device 10. The coil-based vehicle 150 can be formed from a biocompatible polymer, metal, composite, or any other suitable material as would be known to one of ordinary skill in the art. If desired, the coil-based vehicle 50 can be flexible so as to bend in a semi-spiral shape.

The therapeutic agent 52 can be located on or in the removable insert 49 in many forms including but not limited to fluids, gels, solids, suspensions, emulsions, slow-release or time-release beads/microsphere, nanoparticles, capsules, liposomes, cells, tissue, ion-exchange beads, biodegradable polymers, pellets, or other micro/nano-particulate forms.

A removal element 54 can be located at the proximal end 24 of the insert 49. The removal element 54 can be formed from a biocompatible polymer, metal, composite, or any other suitable material as would be known to one of ordinary skill in the art and can be utilized to insert and remove the insert 49 from the body 20.

Any suitable therapeutic agent 52 can be utilized in conjunction with the disclosed devices. Examples of suitable therapeutic agents 52 that can be utilized in the ocular region include but are not limited to antibiotics, antifungals and antivirals such as erythromycin, tetracycline, aminoglycosides, cephalosporins, quinolones, penicilins, sulfonamides, ketoconazole, miconazole, acyclovir, ganciclovir, azidothymidine, vitamins, interferon; anticonvulsants such as phenytoin and valproic acid; antidepressants such as amitriptyline and trazodone; antiparkinsonism drugs; cardiovascular agents such as calcium channel blockers, antiarythmics, beta blockers; antineoplastics such as cisplatin and methotrexate, corticosteroids such as dexamethasone, hydrocortisone, prednisolone, and triamcinolone; NSAIDs such as ibuprofen, salicylates indomethacin, piroxicam; hormones such as progesterone, estrogen, testosterone; growth factors; carbonic anhydrase inhibitors such as acetazolamide; prostaglandins; antiangiogenic agents; neuroprotectants; neurotrophins; growth factors; cytokines; chemokines; cells such as stem cells, primary cells, and genetically engineered cells; tissues; and other agents known to those skilled in the art to benefit from controlled or sustained release from implantable devices or combinations thereof.

Representative therapeutic agents 52 that can be used to treat otic tissues include but are not limited to urea, mannitol, sorbitol, glycerol, lidocaine, xylocaine, epinephrine, immunoglobulins, sodium chloride, steroids, heparin, hyaluronidase, aminoglycoside antibiotics (streptomycin/gentamnycin), antioxidants, vitamin, neurotrophins, growth factors, cytokines, chemokines, various therapeutic peptides, polysaccharides, cells such as stem cells, primary cells, and genetically engineered cells as well as other tissues. In some embodiments, glial-cell derived neurotrophic factors can be utilized. Likewise, the treatment of inner ear tissues and/or fluids can involve altering the pressure, volumetric, and temperature characteristics thereof. A precise balance must be maintained in connection with the pressure of various fluids inside the inner ear and its associated compartments. Imbalances in inner ear fluid pressure levels can cause numerous problems.

In those embodiments in which the therapeutic agent 52 is delivered from the body 20 via utilization of a insert 49, either during or after delivery of therapeutic agent 52 to the treatment area, the insert 49 can be removed and refilled for further delivery of additional therapeutic agent 52. The therapeutic agent 52 can be adjusted or changed in accordance with the goals of treatment for a particular condition.

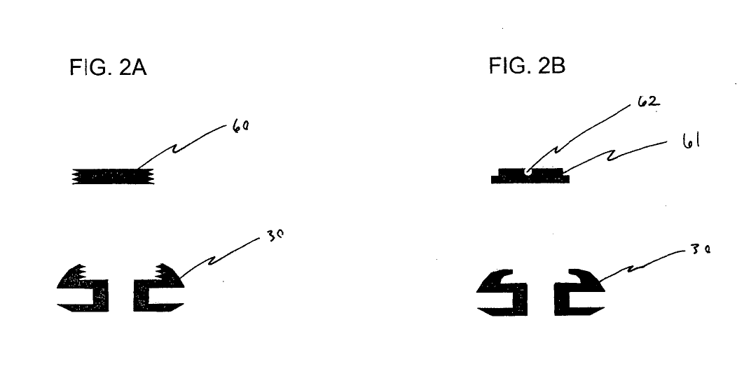

As illustrated in FIGS. 2A and 2B, in some embodiments, the re-sealable access port 30 can have a re-accessible cap 60, 61 which can allow the insert 49 to be retrieved from the lumen 26 of the delivery device 10. The overall size and shape of the cap 60, 61 is not particularly limited. However, the cap 60, 61 can, in certain preferred embodiments, and as shown in FIGS. 1A and 1B, complement the re-sealable access port 30 such that therapeutic agent 52 can be held within the device and exit from proximal end 24 of the body 20 is prevented.

FIGS. 2A and 2B schematically illustrate two exemplary embodiments of a cap 60, 61, as may be utilized in accordance with certain embodiments of the present disclosure. For example, as illustrated in FIG. 2A, the cap 60 can be thread based, while in other embodiments, as illustrated in FIG. 2B, the cap 61 can plug-in to the re-sealable access port 30. Thus, the insert 49 and cap 60 can allow for easy retrieval as well as easy substitution or refilling of therapeutic agent within the device 10.

Referring to FIG. 2B, in some embodiments, the cap 61 can have a port 62 through which a portion of the coil-based vehicle 50 can be exposed. The port 62 can form a tight seal about the insert 49 to prevent leakage of therapeutic agent 52 out of the lumen 26 through the port 62. The exposed portion of the insert 49 can aid in retrieval by allowing the insert 49 to be pulled more easily from the delivery device 10.

The delivery device 10, of the present disclosure can be used for controlled, sustained release of therapeutic agent 52 for treating a variety of ocular diseases and otic diseases.

In this regard, delivery refers to the release of a therapeutic agent from the delivery device 10 such that the therapeutic agent 52 is delivered into an environment surrounding the delivery device 10. The environment into which the therapeutic agent 52 is released can be the ultimate site of activity for that therapeutic agent 52, though this is not a requirement of the present disclosure. In some instances, for example, the released therapeutic agent can be transported to its ultimate site of activity, for instance via the blood stream or any other suitable natural biological activity.

The delivery device 10 of the present disclosure can be used for treating ocular diseases such as, for example, retinal degeneration, retinal detachment, proliferative retinopathy, proliferative diabetic retinopathy, degenerative disease, vascular diseases, occlusions, infection caused by penetrating traumatic injury, endophthalmitis such as endogenous/systemic infection, post-operative infections, inflammations such as posterior uveitis, retinitis or choroiditis, tumors such as neoplasms and retinoblastoma, cataract, and secondary nerve degeneration. Many of theses diseases can be beneficially treated with the device due to the long-term intraocular delivery of therapeutic agents possible with the disclosed devices.

Similarly, the delivery device 10 of the present disclosure can be used to treat various diseases and conditions associated with the inner ear including deafness, sensorineural hearing loss, autoimmune inner ear disease, Meniere's disease, tinnitus, otitis, otalgia, and other otic diseases.

Methods of implanting the delivery device 10 are well-known in the art, and can include surgical means, injection, trocar, or the like.

For example, with specific regards to the ocular region, and as illustrated in FIG. 7, in one particular embodiment, the delivery device 10 can be placed substantially upon the outer surface of the eye and can be anchored in the conjunctiva or sclera, or episcierally or intrasclerally over an avascular region. The delivery device 10 can also be implanted substantially within the suprachoroidal space over an avascular region such as the pars plana or a surgically-induced m avascular region. Of course, any other suitable implantation site is encompassed by the present disclosure.

For example, in another embodiment, the delivery device 10 can be implanted in an area in direct communication with the vitreal chamber or vitreous so as to avoid diffusion of the drug into the bloodstream. The delivery device 10 can optionally be implanted in the anterior chamber. In yet another embodiment, diffusion of the therapeutic agent 52 to the desired site can be facilitated by forming communicating channels e.g., holes or tunnels, through the layers of the sclera or other tissue which communicate, with the desired site of therapy which lie beneath the delivery device 10. According to such an embodiment, the tunnels can lie beneath the implant and serve to direct the flow of therapeutic agent 52 from the delivery device 10 to the desired site of therapy. Alternatively, the delivery device 10 can be inserted so as to directly communicate with the vitreal chamber. For example, a hole of suitable size can be made through the sclera to communicate with the base of the vitreous body through the pars plana. The delivery device 10 can then be positioned over the hole within the scleral bed and the flap of the hole sewn back into place. Such placement of the delivery device 10 can allow for the ready diffusion of the drug into the vitreous and into the intraocular structure.

With regard to the otic region, numerous devices can be utilized (see e.g., FIGS. 5A, 5B, and 6D). For example, in one embodiment as depicted in FIGS. 5A and 5B, a device can be utilized for patients already having a cochlear implaint.

In one embodiment depicted in FIG. 6D, the delivery device 110 can be incorporated into a new cochlear implant. As illustrated in FIGS. 6A and 6D, a cochlear implant 72 incorporating a delivery device 110 of the present disclosure is shown. A portion of the body 120 of the delivery device 110 can be exposed to tissue and therapeutic agents 52 can be released to surrounding tissue. FIG. 6D illustrates a cross section of a cochlear implant 72 and delivery device 110 with therapeutic agents 52 being released from the delivery device 10. For instance, the delivery device 10 can be incorporated into a cochlear implant 72 such that device is inserted inside the cochlea 70. It has been found that scar suppression agents are preferably delivered at the early stages of implantation (1-14 days) and aid in the eventual life-time delivery of neurotrophins using genetically engineered cells.

These and other modifications and variations to the present disclosure can be practiced by those of ordinary skill in the art, without departing from the spirit and scope of the present disclosure, which is more particularly set forth in the appended claims. In addition, it should be understood that aspects of the various embodiments can be interchanged both in whole or in part. Furthermore, those of ordinary skill in the art will appreciate that the foregoing description is by way of example only and is not intended to limit the disclosure so further described in such appended claims.

What is claimed is:

1. A device for delivery of therapeutic agents comprising:
a body, said body including a proximal end and a distal end, said body defining a cavity;
an access port, said access port located at said proximal end of said body; and
a removable insert, said removable insert having a proximal end and a distal end, said removable insert configured to be removably inserted into said cavity of said body.

2. The device as defined in claim 1, wherein said removable insert further includes one or more therapeutic agents.

3. The device as defined in claim 1, wherein said body is configured for use in proximity to the ear.

4. The device as defined in claim 2, wherein said body is configured for use in proximity to the eye.

5. The device as defined in claim 1, wherein said body further comprises a selectively permeable hollow fiber membrane.

6. The device as defined in claim 5, wherein said selectively permeable hollow fiber membrane has less than 200 KDa molecular weight cut off.

7. The device as defined in claim 5, wherein said selectively permeable hollow fiber membrane has less than 70 KDa molecular weight cut off.

8. The device as defined in claim 5, wherein said selectively permeable hollow fiber membrane has less than 40 KDa molecular weight cut off.

9. The device as defined in claim 5, wherein said selectively permeable hollow fiber membrane has less than 20 KDa molecular weight cut off.

10. The device as defined in claim 1, wherein said access port is formed from a biocompatible flexible polymer.

11. The device as defined in claim 2, wherein said access port further comprises a cap, said cap configured to removably engage said access port such that no therapeutic agent can exit said body through said access port.

12. The device as defined in claim 1, wherein said removable insert further comprises a selectively permeable hollow fiber membrane.

13. The device as defined in claim 2, wherein said removable insert further comprises a degradable rod, said degradable rod configured to dissolve and release said therapeutic agent.

14. A method for targeted delivery of therapeutic agent to the inner ear comprising:
providing a device comprising a body, an access port, and a removable insert, said body comprising a selectively permeable hollow fiber membrane, said removable insert comprising one or more therapeutic agents;
implanting said device whereby said therapeutic agent is delivered to an inner ear.

15. The method of claim 14, wherein the therapeutic agent delivered is a neurotrophic agent.

16. The method of claim 14, wherein said access port is a biocompatible flexible polymer.

17. The method of claim 14, wherein said selectively permeable hollow fiber membrane has less than 200 KDa MWCO.

18. A method for targeted delivery of therapeutic agent to the eye comprising:
providing a device comprising a body, an access port, and a removable insert, said body comprising a selectively permeable hollow fiber membrane, said removable insert comprising one or more therapeutic agents;
implanting said device whereby said therapeutic agent is delivered to an eye.

19. The method of claim 18, wherein the therapeutic agent delivered is an antibiotic.

20. The method of claim 18, wherein said access port is a biocompatible flexible polymer.

21. The method of claim 18, wherein said selectively permeable hollow fiber membrane has less than 200 KDa MWCO.
